(19) United States
(12) Patent Application Publication
(10) Pub. No.: US 2005/0182320 A1
(43) Pub. Date: Aug. 18, 2005

(54) Arrangement for ascertaining function-determining geometric parameters of a joint of a vertebrate

(76) Inventors: Jan Stifter, Unterehrendingen (CH); Holger Broers, Uplengen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/994,186

(22) Filed: Nov. 19, 2004

Related U.S. Application Data
(63) Continuation of application No. PCT/EP03/01635, filed on Feb. 2, 2003.

(30) Foreign Application Priority Data
May 21, 2002 (DE) 102 22 416.1

(51) Int. Cl. A61B 5/05

(52) U.S. Cl. 600/429

(57) ABSTRACT

Arrangement for ascertaining function-determining geometric parameters of a joint of a vertebrate, especially a hip or shoulder joint of a human being, in preparation for the installation of a joint replacement implant, especially a hip or shoulder socket or an associated stem implant, by means of an optical coordinate-measuring procedure, having a stereocamera or stereocamera arrangement for the spatial recording of optical transducer signals, a mobile multipoint transducer which is in the form of a movable sensor for sensing bony references in the joint region in order to determine the coordinates thereof, at least one bone-fixed multipoint transducer which is configured for rigid attachment, especially screwed or clamped attachment, (in a region sufficiently distant from the joint) to an extremity originating from the joint, especially close to the proximal end of a femur or a humerus, an interactive sequence controller for controlling the sequential registration and storage of a set of measurement point coordinates supplied by the mobile multipoint transducer and sets of measurement point coordinates recorded in a first plurality of positions of the bone-fixed multipoint transducer in a plurality of rotated positions of the extremity and their subsequent processing in accordance with a previously stored processing sequence, an evaluation unit for evaluating the sets of measurement point coordinates supplied by the multipoint transducers and recorded by the camera arrangement for the purpose of determining the geometric parameters, which comprises means for determining the transversal, vertical and sagittal body axes as well as means for carrying out an iterative procedure, especially an adjustment calculation in accordance with the least squares method, to determine the coordinates of the center of rotation of the joint, and an output unit, which is connected to the sequence controller and to the evaluation unit, for issuing manipulation proposals to an operating surgeon in accordance with the predetermined process sequence and in dependence upon the results of the determination of the geometric parameters, and for displaying the results of the evaluation.

### RELATED APPLICATIONS

This is a Continuation of PCT application PCT/EP03/01635, which was filed Feb. 2, 2003 and published in German on Nov. 27, 2003 as WO 03/096920, and which is incorporated herein by reference. The above PCT application claims priority to German patent application Serial No. 102 22 416.1, filed May 21, 2002.

### BACKGROUND

1. Field of the Invention

The invention relates to an arrangement for ascertaining function-determining geometric parameters of a joint of a vertebrate and also to a corresponding method.

2. Description of the Related Art

Surgical interventions for the replacement of joints or joint components in human beings have been known for a long time and form part of everyday clinical procedure in industrialized countries. For decades, intensive development work has also been carried out with a view to the provision and continuing improvement of such implants, especially hip joint implants but increasingly also knee, shoulder and elbow joint implants as well as vertebral replacement implants. In parallel with those developments, which have now resulted in an almost infinite variety of such implant structures, there are also being made available and further developed suitable operating techniques and aids, including, especially, tools for the installation of implants that are matched to the implant structures in question.

It will also be understood that joint replacement operations are preceded by the acquisition of suitable images of the joint region in question, on the basis of which the operating surgeon determines a suitable implant and the surgical technique. Whereas formerly X-ray images were generally used for this purpose, in recent years computer tomograms have also become the tool of the operating surgeon. Nevertheless, the long-term success of joint replacement implantations is even today still closely associated with the experience of the operating surgeon, and this must to a considerable extent be attributed to the difficulties, which are not to be underestimated, of appropriate intra-operative utilization of visual images for achieving optimum alignment of the components of the joint implant in relation to the effective joint centers and load axes of the individual patient.

In recent years, therefore, there have been increased efforts to provide suitable positioning aids and methods for the operating surgeon, which have been derived substantially from developments in the field of robotics and manipulation techniques.

EP 0 553 266 B1 and U.S. Pat. No. 5,198,877 describe a method and an apparatus for contactless three-dimensional shape detection, which has provided stimulus for the development of medical “navigation” systems and methods; see also the detailed literature references in those specifications.

U.S. Pat. No. 5,871,018 and U.S. Pat. No. 5,682,886 disclose methods of ascertaining the load axis of the femur. In accordance with those methods, in a first step the coordinates of the femur are ascertained, for example by means of a computer tomography image, and stored in a computer. The stored data are then used to create a three-dimensional computer model of the femur and, with the aid of that model, the optimum coordinates are calculated for the positioning of a jig on the bone and of a knee prosthesis that is subsequently to be installed. The basis for this is the calculation of the load axis of the femur.

After such a simulation, the patient's femur is fixed in position and, using a registration device, contact is made with individual points on the femur surface in order to establish the orientation of the femur for the operation to be carried out. Such contacting of the bone requires either that the femur be exposed along large portions of its length, if possible as far as the hip joint, in order that its surface can be contacted with the registration device or that a kind of needle be used as a probe for penetrating through the skin as far as the bone. Since, however, any surgical intervention constitutes a risk to the patient and needle pricks cause bleeding and an additional risk of infection in the region of the bones, it is undesirable to perform an additional surgical intervention in the hip region or to insert needles along the femur in order to establish the location of the center of rotation. Furthermore, the femur needs to be firmly fixed on the measurement table of a registration device, because otherwise the hip socket may become displaced during the probing procedure, with the possibility that, once the registration of the femur coordinates is complete, the cutting jig will be incorrectly positioned.

FR 2 785 517 describes a method and a device for detecting the center of rotation of the head of the femur in the hip socket. For this purpose, the femur is moved with its head in the hip socket and the measurement point coordinates recorded in various positions of the femur are stored. As a soon as a shift in the center of rotation of the femur occurs, a corresponding counter-pressure is exerted on the head of the femur, which is taken into account in the determination of a point which relates to the arrangement of the femur.

DE 197 09 960 A1 describes a method and a device for the pre-operative determination of position data of endoprosthetic components of a central joint relative to the bones forming the central joint, it being proposed that an outer articulation point be determined by moving each of the bones about an outer joint located at the end of the bone in question that is remote from the central joint; that in the region of the said central joint an articulation point likewise be determined for each of the two bones; that by joining with a straight line the two articulation points so found for each of the two bones there be determined a direction characteristic thereof and finally that the orientation of the endoprosthetic components relative to that characteristic direction be determined.

Similar medical “navigation” methods are described in WO 95/00075 and WO 99/23956 wherein image-acquisition systems of the kind mentioned above are used for recording the position of references on the bones adjacent to the joint in question and characteristic points and axes can be derived from the virtual representation of the bone or joint obtained by that means.

A system of that kind, which has been improved in respect of reliability and, especially, in respect of independence from intra-operative movements of the patient and which is intended for direct use during surgery, especially the implantation of an artificial knee joint, is the subject of the Applicant's specification WO 02/17798 A1.

### SUMMARY

Starting from the prior art, the invention is based on the problem of providing an arrangement of that kind which is quickly and easily operated by the operating surgeon with a very low risk of error and which enables significantly improved surgical results to be achieved, especially in the case of hip and shoulder joint implants.

This problem is solved in terms of apparatus by an arrangement having the features of claim 1 and in terms of method by a method having the features of claim 11. The subsidiary claims relate to advantageous variants of the inventive concept. Their subject matter, in any combination with one another, including modifications, lie within the scope of the present invention.

A basic concept of the invention lies in configuring the proposed arrangement for ascertaining function-determining geometric parameters of a joint in preparation for a joint replacement implantation with a stereocamera or stereocamera arrangement and two different kinds of signal transmitters therefor. The latter includes (at least) one first (“mobile”) multipoint transducer which is in the form of a movable sensor for sensing bony references in the joint region in order to determine the coordinates thereof, and a second (“bone-fixed”) multipoint transducer which is configured for rigid attachment, especially screwed or clamped attachment, to an extremity originating from the joint in a region sufficiently distant from the joint, especially close to the proximal or distal end of a femur or a humerus.

The invention also includes the concept of providing an interactive sequence controller for controlling the sequential registration and storage of sets of measurement point coordinates recorded in a first plurality of sensor positions of the first multipoint transducer and a second plurality of rotated positions of the extremity and their subsequent processing in accordance with a predetermined processing sequence.

Lastly, the invention includes the concept of providing a suitably configured evaluation unit for evaluating the set of measurement point coordinates supplied by the multipoint transducers and recorded by the camera arrangement for the purpose of determining the geometric parameters. The evaluation unit comprises means for determining the transversal, vertical and sagittal body planes and axes as well as means for carrying out an iterative procedure, especially an adjustment calculation in accordance with the least squares method, to determine the coordinates of the center of rotation of the joint.

Finally, the arrangement according to the invention includes an output unit, which is connected to the sequence controller and to the evaluation unit, for issuing manipulation proposals to an operating surgeon in accordance with the predetermined process sequence and in dependence upon the results of the determination of the geometric parameters, and for displaying the results of the evaluation.

The said output unit is advantageously configured for displaying the results of the evaluation in graphic form, especially in a synoptic visual display with a two-dimensional or three-dimensional image of the joint region obtained by an imaging test procedure. As a result—independently of an interactive user guidance system advantageously implemented in the system and automatic control functions—the operating surgeon has a good opportunity of obtaining a visual impression of the geometric relationships in the joint region and, where applicable, of the position of a tool or of the implant relative thereto.

Specifically for a socket implantation (in the hip or shoulder region) there is also used a further bone-fixed multipoint transducer which is rigidly attached to a bony region on the socket side of the joint (for example on the iliac crest) and the position signals of which in conjunction with those of the mobile sensor serve for socket-side position determination.

A feature important for the broad practical use of the proposed arrangement is an input interface for entering position reference vectors between defined real or virtual points of the joint region and/or position reference vectors between such points within the joint region or from those points to joint-function-relevant points on the extremity outside the joint region and/or implant parameters of a predetermined set of suitable joint replacement implants or for specifying possible implant positions and alignments, the interface being connected to the sequence controller and to the evaluation unit. Such an interface is either a user interface for keyboard entry or voice entry of data by the operating surgeon or an interface for transferring data from an evaluation program based on an imaging test procedure or an interface that combines those functions with one another.

The arrangement advantageously includes at least one adjustable clamping device as an adapter for fixing the bone-fixed multipoint transducer in position on the extremity or for fixing the multipoint transducers to extremity and joint, or an appropriate mounting device based on screws or nails anchored in bone.

Furthermore, the mobile multipoint transducer is configured for the external sensing of bony references on the second extremity originating from the joint being replaced and, as desired, from the second hip or shoulder joint, or alternatively a further multipoint transducer in the form of a movable sensor for sensing such references is provided for that purpose. The evaluation unit is in that case configured for evaluating the measurement point coordinates of those bony references in order to determine at least one of the geometric parameters, especially the length of the extremity.

It is also advantageous to supplement the arrangement with a third (bone-fixed) multipoint transducer for substantially rigid attachment, especially by means of an adjustable sleeve, to a second extremity which originates from a second hip or shoulder joint that is not undergoing surgery. In that case the evaluation unit enables geometric parameters of the second hip or shoulder joint to be determined as a reference for the geometric parameters of the first joint.

An integrated total arrangement of the kind according to the invention preferably also comprises a resectioning instrument, especially a milling tool or a rasp, for shaping the implantation region and/or a navigable setting instrument, especially a screwing tool, for mounting the joint replacement implant. A further multipoint transducer is provided in association with one or both of those tools, or the mobile multipoint transducer mentioned above is used therewith. It can be rigidly connected to the tool in question to form a geometrically calibrated, navigable tool/transducer unit, so that from the transducer signals of that unit there can be determined position coordinates of an operational part of the instrument, and therefrom, as desired, position coordinates of a resection zone produced with the resectioning instrument or of the implant. In this case the input interface is configured especially for entering instrument parameters of the resectioning instrument and/or tool parameters of the setting instrument.

In a further advantageous development of the inventive concept, the arrangement comprises a probe, especially a medullary canal awl, for probing the medullary canal of the extremity originating from the joint, which probe can be rigidly connected to a multipoint transducer to form a geometrically calibrated, navigable probe/transducer unit, so that the transducer signals of that unit can be used to determine a direction vector of the medullary canal. It will be understood that in this case the input interface must be suitable for entering probe parameters.

The multipoint transducer(s) is(are) preferably in the form of passive four-point transducers having four spherical reflector parts. The stereocamera or camera arrangement is associated with an illuminating device with which the multipoint transducer(s) are illuminated, so that defined reflections for “imaging” the multipoint transducer in question are available. In order to avoid light reflections that would disturb the operating surgeon, the illuminating device preferably operates in the infrared range.

A variant of the proposed arrangement that provides especially extensive support for the operating surgeon comprises a control signal generation unit that is connected to the evaluation unit and to the matching-processing unit. This is configured for comparing a set of implant position data or alignment data that has been entered by means of the input interface and matched to the real position coordinates of the joint region or vertebral region with currently acquired real position coordinates of the operational part of the resectioning instrument or setting instrument and for determining any variance between desired position and actual position coordinates and for outputting variance data or a control command derived from the variance, especially by means of a text or speech output and/or in a synoptic display with the image.

As regards the method aspects of the invention, they correspond substantially to the apparatus aspects discussed above, reference being made expressly thereto.

### BRIEF DESCRIPTION OF DRAWINGS

Advantages and useful features will otherwise be found in the following description of a preferred embodiment—an arrangement in connection with a method for the implantation of an artificial hip joint—in conjunction with the Figures, in which:

FIG. 1 shows a perspective view of an iliac crest locator having an associated clamp (adapter) clamped onto an iliac crest;

FIG. 2 additionally shows a perspective view of a manual sensor for sensing the table surface for the purpose of determining the table plane as well as bony references on the iliac crest (though the skin);

FIG. 3 shows, in addition to the iliac crest locator, a perspective view of a femur locator having an associated clamp for fixation in the proximal region of a femur;

FIG. 4 shows a perspective view of a sphere adapter/manual sensor combination for determining the center of the acetabulum;

FIG. 5 shows a perspective view of a milling tool/locator combination for milling the seat for a hip socket;

FIG. 6 is a diagrammatic detail view of the display of a PC monitor for visually displaying views of the milling tool relative to the pelvis;

FIG. 7 is a perspective view of a setting instrument/locator combination for screwing an artificial hip socket into the prepared seat, and

FIG. 8 is a perspective view of a medullary canal awl/locator combination for determining the path of the medullary canal in a femur.

### DETAILED DESCRIPTION

The following description is given primarily with reference to a procedure for determining the relevant geometric parameters and for implanting a hip socket, but reference is additionally made also to the determination (relatively independent thereof) of the relevant geometric parameters and the implantation of a stem component as the second component of an artificial hip joint.

The operating surgeon, when planning a hip joint implantation, needs to determine the following values for the socket:
1. Size of the artificial socket
2. Angle of inclination and antetorsion angle
   The two angles of alignment of the socket axis relative to the body planes are here selected on an X-ray image by the operating surgeon in accordance with medical standpoints. These angles can likewise be modified by the operating surgeon intra-operatively.
3. Angle in the sagittal body plane between vertical axis and the direction from the iliac crest to the symphisis.
   Determining this angle allows intra-operative determination of the body axes and thus of the plan coordinate system.

It is assumed that the patient is supine at the beginning of the operation; the physician has an X-ray image available which gives an adequate picture of the overall anatomical situation and the nature of the bones and from which he makes his first deductions as to the size of implant to be installed and the preferred approximate alignment of the implant. An incision, 4 cm in length, is made 3-5 cm dorsally of the spina iliaca superior anterior, the iliac crest is exposed and the tissue is exposed with a rasp.

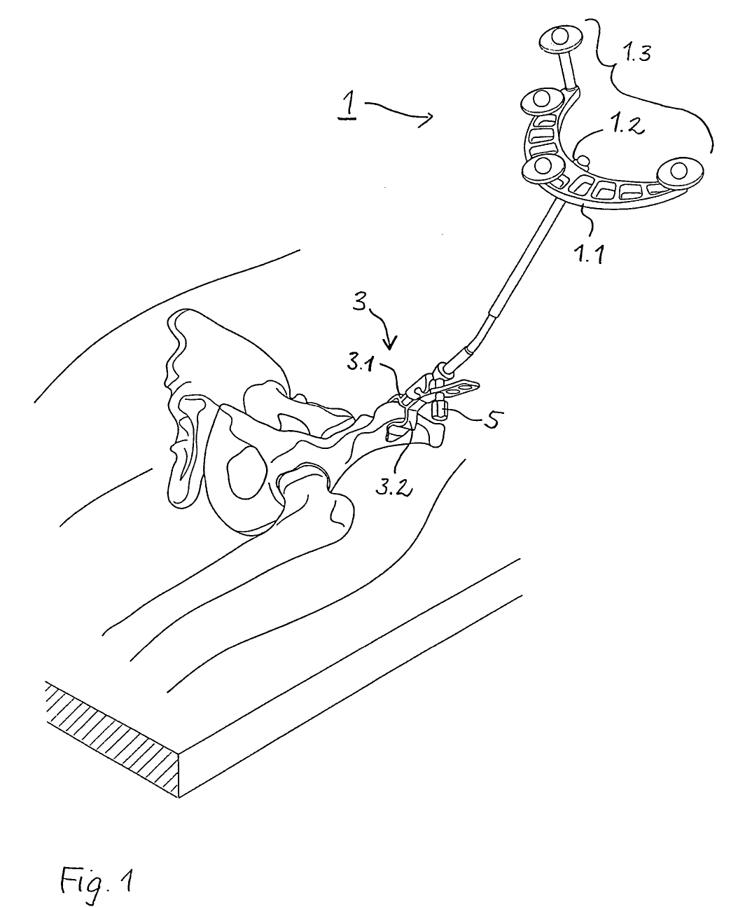

FIG. 1 shows an iliac crest locator 1 with an associated mounting clamp 3, which is attached in the exposed region of the iliac crest. The mounting clamp 3 comprises a medial clamp component 3.1 and a lateral clamp component 3.2, which are screwed together by means of an Allen bolt 5 until the mounting clamp is firmly seated on the iliac crest. The actual iliac crest locator 1 has a sickle-shaped basic body 1.1 having a mounting sleeve 1.2 for positioning on the mounting clamp 3 as well as a 4-point locator array 1.3 consisting of four IR-reflecting spheres each of which is partially surrounded by a diffuser (not separately referenced) in the shape of a spherical segment in order to avoid troublesome radiation effects. These are so-called passive targets or adapters which are known per se and the mode of operation of which in conjunction with the (likewise known) stereocamera arrangement of a so-called navigation system will therefore not be described in greater detail here. After being put in position, the locator 1 is rotated relative to the mounting clamp 3 so that the locator array is suitably aligned relative to the camera but without any of the reflecting spheres being masked by another one. Then, by screwing the locator and the mounting clamp together, a rigid connection is established between the two.

Instead of being attached to the iliac crest, the multipoint transducer 1, referred to as the iliac crest locator above, can also be attached to the roof of the acetabulum of the pelvis. This has the advantage that the above-mentioned (additional) incision in the region of the iliac crest becomes superfluous, but the attachment of the multipoint transducer, which is then referred to as the “surgical field locator”, is less stable if the bone structure is weak.

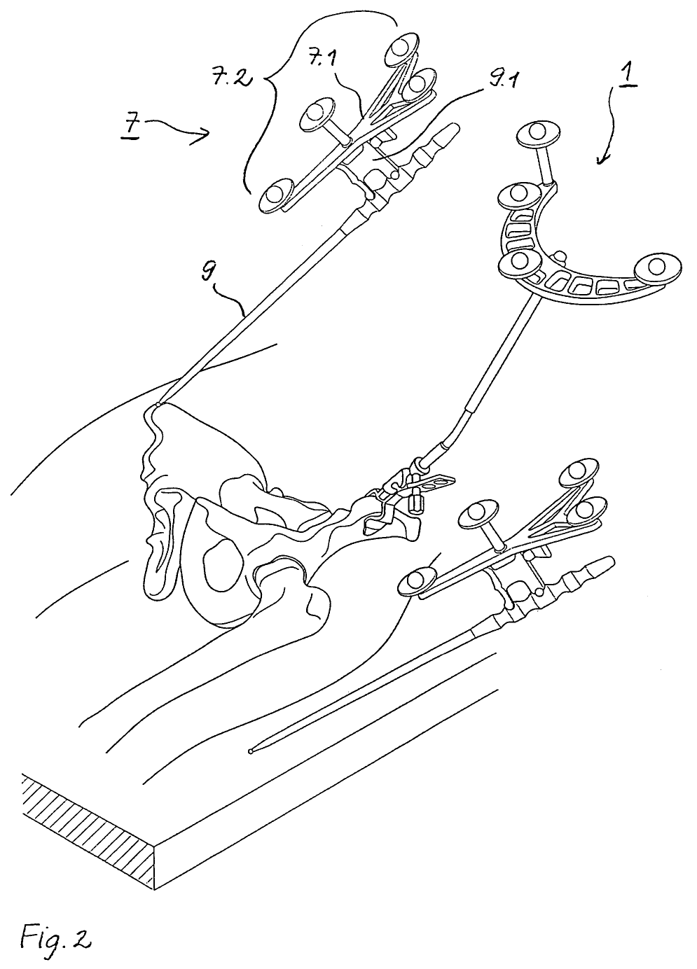

FIG. 2 shows, in addition to the above-described bone-fixed locator 1, a manual sensor 7 having a rod-shaped sensing component 9, which tapers towards one end and from which a holder 9.1 projects perpendicularly, an approximately Y-shaped sensor body 7.1 and a 4-point locator array 7.2, similar to the structure of the iliac crest locator described above. The locators of the components of the arrangement described below are also of similar structure, so that the naming of the corresponding parts and portions of those locators and the description thereof will be omitted.

Using the manual sensor 7, at the beginning of the navigation sequence various points on the plane of the operating table on which the patient is lying are scanned in order to determine the position of the table plane in space. Although this is not required for the actual determination of the patient's position, it can be used for plausibility considerations (for example in respect of the significance of the inclination of the patient's pelvis relative to the plane of the table etc.). For the actual navigation it is usually assumed that the patient's frontal plane lies parallel to the plane of the table.

Then, using the manual sensor 7, characteristic bony references in the pelvis region are sensed through the skin. First of all, the left and right iliac crests and the center of the symphysis are sensed. These three sensed points and the crest/symphysis angle ascertained during the planning enable the body axes to be clearly determined. The direction from left iliac crest to right iliac crest represents the transversal body axis. The direction from the center of the iliac crest points to the symphysis is rotated through the crest/symphysis angle about the transversal axis and thus represents the vertical body axis (orthogonal to the transversal axis). The sagittal body axis is obtained from the two first-mentioned axes as an orthogonal.

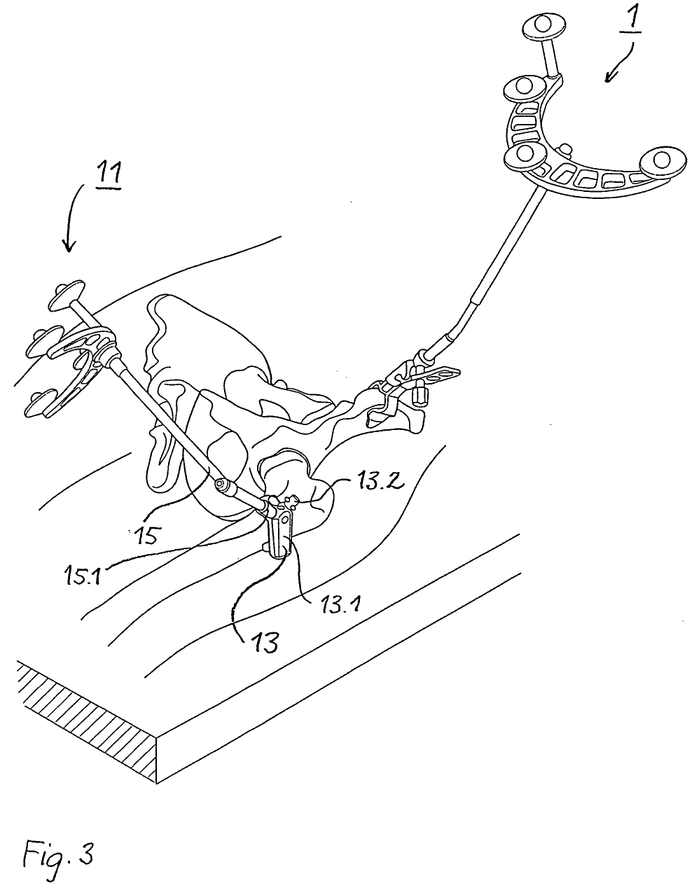

FIG. 3 shows, in addition to the iliac crest locator 1, a femur locator 11 having an associated adapter (femoral clamp) 13 for attachment close to the proximal end of the femur. The femoral clamp 13 has a two-part body consisting of a first base member 13.1, which is fork-shaped in plan view and approximately L-shaped in side view, from which two pins 13.2 project for mounting the locator, and a second base member, which is approximately L-shaped in side view and which can be locked together with the first base member 13.1. The structure of the femur locator 11 itself, apart from having an angled locator rod, is substantially the same as that of the iliac crest locator.

It is pushed by way of a mounting sleeve 15.1 at the free end of a locator rod 15 onto one of the two pins 13.2 of the femoral clamp 13.

The femoral clamp 13 is then attached to the mounted locator rod 15 on the lateral femur side approximately at the level of the trochanter minor or between the trochanter minor and the trochanter major, by pushing the muscle groups located there aside and inserting the clamp. The rotated position is to be so selected that the locator rod projects laterally out of the surgical field, if possible in the direction of the camera. Then the clamp is tightened with a moderate torque, the actual locator array (not separately referenced here) is mounted and aligned towards the camera and finally the femur locator is screwed tight.

The kinematic center of rotation of the hip is then determined both in the hip-fixed coordinate system and in the femur-fixed coordinate system by a plurality of relative measurements of the femur locator in the hip-fixed coordinate system with the leg in different positions. The transformation of all measured values can accordingly be effected from the hip-fixed coordinate system into the coordinate system of the body axes. Accordingly all the calibrated tools can then be aligned relative to the body axis coordinate system; in this connection see below. Using the center of rotation as origin, the implant can be installed at its kinematic origin. Should corrections be necessary, displacements and changes of angle in the plan can be carried out intra-operatively.

Once the operating surgeon has carried out the position recordings in the various positions of the leg in “dialogue” with the interactive user guidance (error correction again being provided on the basis of plausibility calculations), the femur locator is removed from the clamp 13 and the head of the femur is resectioned. The diameter of the resectioned head is measured and, on the basis of the measurement result, a suitable hemisphere is selected for the next step, namely the determination of the center of the acetabulum or geometric center of rotation of the hip.

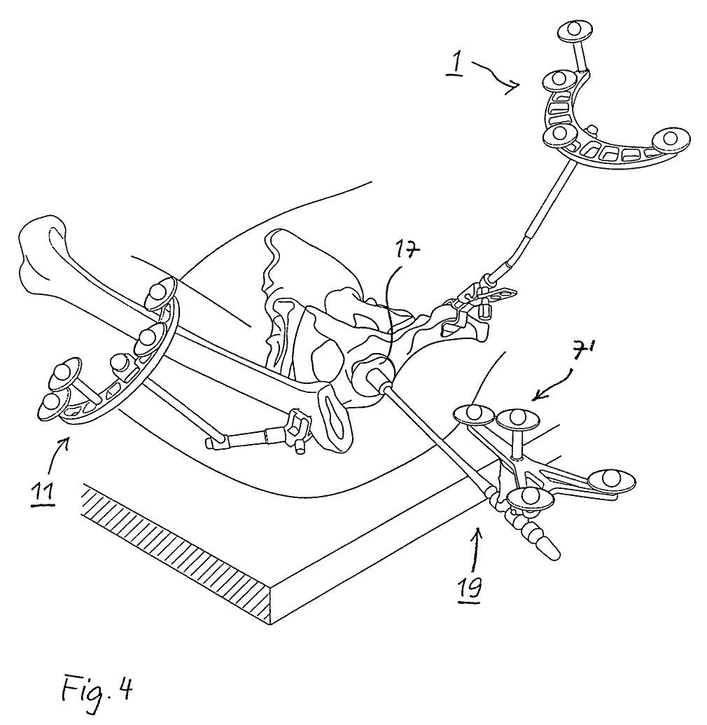

As shown in FIG. 4, the selected hemisphere 17 is combined with a manual sensor 7′ of the kind shown in FIG. 2 and described above to form a sphere adapter/manual sensor combination 19. By guiding such a locator into the socket region (usually assuming a certain anteversion angle, e.g. 12°), first the validity of the (kinematic) center of rotation determined by means of the femur locator is checked from the geometric point of view and secondly the results allow a “cross-check” of the planned implantation values from geometric standpoints. Furthermore, moving the hemisphere 17 in the socket region provides pointers to possible mechanical collisions. The structure of the half-shell and its adaptation to the manual sensor ensures that the probe tip is always in the sphere center of the sensing hemisphere.

There then follows, within the framework of the stored evaluation program with interactive user guidance, the final planning of the implantation, from the determination of the implant size that is to be installed through to displacement values and angle sizes. On that basis and with reference to previously entered specific instrument data, the system calculates desired positions for the resectioning and setting instruments to be used or, more specifically, for their operational parts.

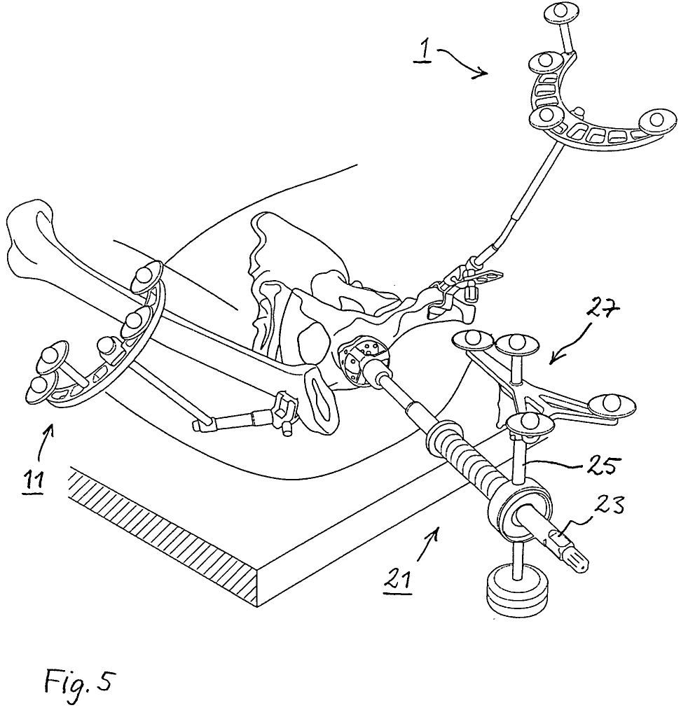

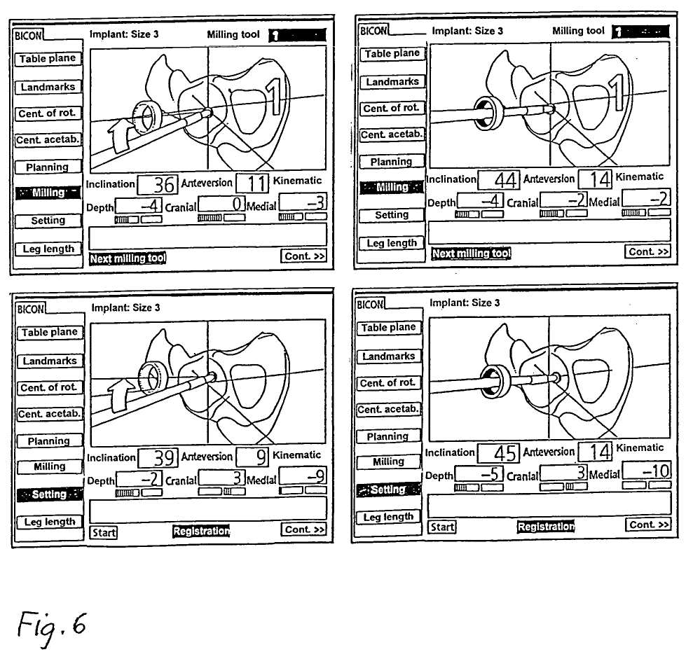

FIG. 5 shows, in addition to the iliac crest and femur locators 1, 11, a milling tool/locator combination 21 having a milling shaft 23, a milling shaft adapter 25 and a locator 27, the structure of which corresponds substantially to that of the femur locator 11 according to FIG. 3. This instrument is aligned in a socket region in the manner likewise shown in the Figure, the position and alignment being recorded on the basis of position signals from the locator array and being displayed visually on screens in the manner shown in FIG. 6. A milling tool position that is correct in accordance with the plan data is indicated on the display by a ring encompassing the milling shaft and by acoustic signals.

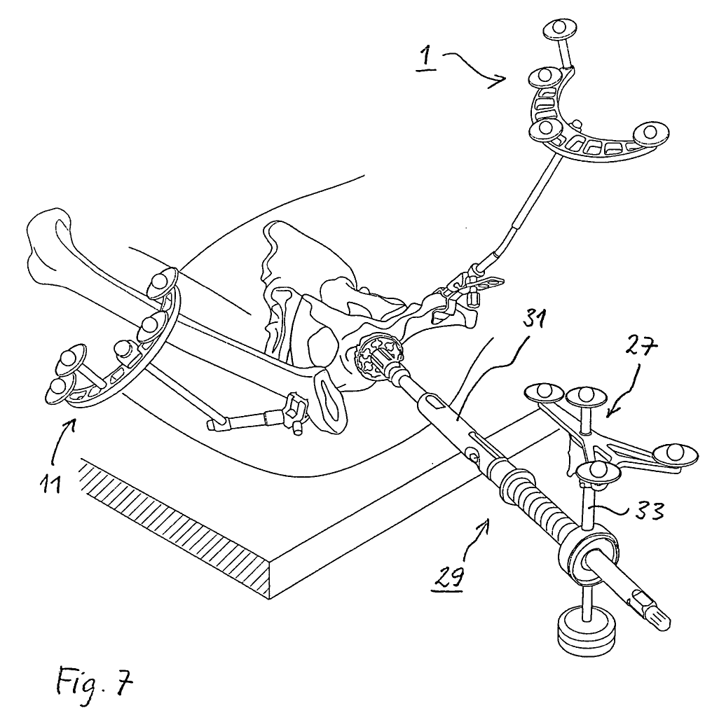

As soon as a socket seat has been produced in accordance with the plan data, the milling tool/locator combination is converted into a setting instrument/locator combination 29, as shown in FIG. 7, the locator 27 again being used but this time in conjunction with a setting instrument shaft 31 and a shaft adapter 33. Using this instrument, a hip socket 35 is set in place in a manner that is largely analogous to the manipulation of the milling tool/locator combination and that is likewise displayed on the PC screen. The ultimate position of the hip socket 35 is still to be entered into the system by the operating surgeon.

Then the stem preparation and implantation (in the first instance a test stem) are carried out, either in a conventional way or again assisted by the navigation system. Height and anteversion of the stem are fixed with reference to the plan data; only the ball neck length is still freely selectable. The joint is then assembled with the test stem, and stability and any potential for collisions during movement of the stem in the socket are tested. In addition, the leg length is roughly tested by comparing the position of the malleoli on the leg undergoing surgery and the healthy leg. If joint stability problems arise, a solution is sought by selecting a specific ball or a stem of a different size from an available range.

Optionally, in this phase it is also possible to take measurements of the other leg using the navigation system, the results of which can be used in the sense of symmetry considerations with a view to fine adjustment of the implant. It will be understood that for such measurements, instead of using the femur locator described above, there is used a femur locator modified for external mounting over the skin.

A considerable advantage of the proposed system is that using navigation data it is also possible to make a “before and after” comparison of the leg lengths (on the diseased hip prior to the operation and during the above-mentioned testing step in the final phase of the operation). For this purpose, the femur locator is again positioned and fixed in place on the holder which has remained on the femur and the position with the leg extended and aligned parallel to the longitudinal axis of the body is recorded. The position data obtained indicate any lengthening or shortening of the leg and also the so-called lateralization or medialization, that is to say the “sided” position of the femur. Where too much metallization (displacement towards the inside) is indicated, a stem different from the test stem can be used in conjunction with a different ball; in any case, however, the measured values suggest to the physician what should be taken into consideration in the further care of the patient.

The following remarks relate to the use of the described system in stem preparation and implantation.

The placement of the stem of a prosthetic hip requires the establishment of a planned antetorsion angle of the femur neck and the creation of the angle of the original leg length. The axial alignment of the stem is governed to a very great extent by the position of the medullary canal in the femur. As a result, it is only therefrom that the actual stem size or its offsets can be calculated.

A calibrated awl is used to determine the medullary canal of the femur. A further important item of information for the placement of the stem is the determination of the center of rotation; see above in this connection.

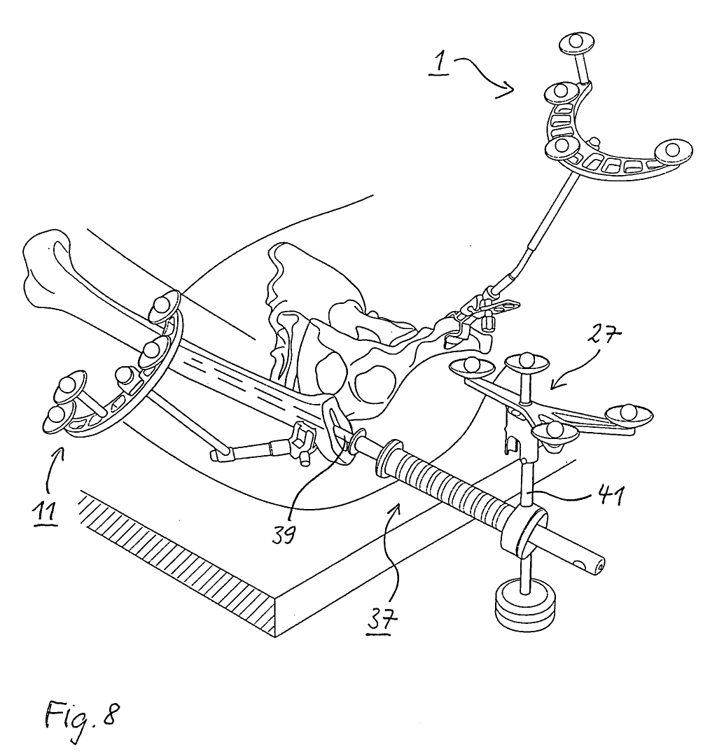

FIG. 8 shows a further component of the proposed arrangement that is suitable for use in this connection, namely a medullary canal awl/locator combination 37 having a medullary canal awl 39, an awl adapter 41 and (again) a locator 27, similar to the locator variant already shown in FIG. 3. For the insertion of this navigation instrument, the proximal femur end is opened with a box chisel or a piercing saw in the vicinity of the trochanter major and the medullary canal awl 39 is inserted therein from the proximal end.

The angle of inclination and antetorsion angle of the head of the femur are determined pre-operatively from an X-ray image and are entered intra-operatively. In addition, the antetorsion angle can be determined intra-operatively by measuring landmarks on the knee joint and on the ankle joint, so that the body planes are known intra-operatively. The actual implantation angles and positions of the socket navigation can also be taken into account in the stem implantation. The last spatial position of the socket can be applied as a relative correction of the stem. This procedure ensures optimum implantation.

The preparation of the femur for installation of the stem is then effected—analogously to the preparation of the socket seat with a navigated milling tool—with a navigated stem rasp, that is to say a stem rasp/locator combination, which is very similar to the combination shown in FIG. 8 and is therefore neither shown nor described in greater detail here. After the preparation, a test stem is again inserted and the tests described above in connection with the socket-side navigation are carried out. When satisfactory results have been obtained, the final stem is then installed without it having to be navigated again.

The invention is not limited to the arrangement described above and the procedure outlined in connection therewith, but can also be realized in modifications that lie within the scope of technical action.

### LIST OF REFERENCE NUMERALS

1 iliac crest locator
1.1 basic body
1.2 mounting sleeve
1.3 4-point locator array
3 mounting clamp
3.1 medial clamp component
3.2 lateral clamp component
5 Allen bolt
7; 7′ manual sensor
7.1 sensor body
7.2 4-point locator array
9 sensing component
9.1 holder
11 femur locator
13 femoral clamp
13.1 first base member
13.2 pin
13.3 second base member
15 locator rod
15.1 mounting sleeve
17 hemisphere
19 sphere adapter/manual sensor combination
21 milling tool/locator combination
23 milling shaft
25 milling shaft adapter
27 locator
29 setting instrument/locator combination
31 setting instrument shaft
33 shaft adapter
35 hip socket
37 medullary canal awl/locator combination
39 medullary canal awl
41 awl adapter

1. An arrangement for ascertaining function-determining geometric parameters of a joint selected from the group consisting of a joint of a vertebrate, a hip joint of a human being, and a shoulder joint of a human being, in preparation for the installation of an implant selected from the group consisting of a joint replacement implant, a hip socket, a shoulder socket and an associated stem implant, by means of an optical coordinate-measuring procedure, said arrangement comprising:
a stereocamera or stereocamera arrangement for the spatial recording of optical transducer signals;
a mobile multipoint transducer, said transducer being in the form of a movable sensor operative to sense bony references in the joint region in order to determine coordinates thereof;
a first bone-fixed multipoint transducer configured for attachment selected from the group consisting of rigid attachment, screwed rigid attachment and clamped rigid attachment, to an extremity selected from the group consisting of an extremity originating from the joint, an extremity originating from a joint adjacent a proximal end of a femur, and an extremity originating from a joint adjacent a proximal end of a humerus;
an interactive sequence controller operative to control sequential registration and storage of a set of measurement point coordinates supplied by the mobile multipoint transducer and sets of measurement point coordinates recorded in a first plurality of positions of the first bone-fixed multipoint transducer in a plurality of rotated positions of the extremity and their subsequent processing in accordance with a previously stored processing sequence;
an evaluation unit operative to evaluate sets of measurement point coordinates supplied by the mobile and first bone-fixed multipoint transducers and recorded by the camera arrangement so as to determine geometric parameters, said evaluation unit comprising means for determining transversal, vertical and sagittal body axes as well as means for carrying out a procedure selected from the group consisting of an iterative procedure, and an iterative adjustment calculation in accordance with the least squares method, to determine coordinates of a center of rotation of the joint, and
an output unit connected to said sequence controller and to said evaluation unit, operative to issue manipulation proposals to an operating surgeon in accordance with the predetermined process sequence and in dependence upon results of the determination of the geometric parameters, and operative to display results of the evaluation.

2. The arrangement as set forth in claim 1, further comprising a second bone-fixed multipoint transducer configured for attachment in a manner selected from the group consisting of rigid attachment, screwed attachment, and rigid clamped attachment, to a region selected from the group consisting of a bony socket-side region of the joint, an iliac crest, and a roof of an acetabulum of a pelvis, wherein said interactive sequence controller is also configured to control the registration and storage of a set of measurement point coordinates supplied by said second bone-fixed multipoint transducer and said evaluation unit is configured for the evaluation thereof.

3. The arrangement as set forth in claim 1, wherein the output unit is configured to display the results of the evaluation in a form selected from the group consisting of graphic form and a synoptic visual display with a two-dimensional or three-dimensional image of the joint region obtained by an imaging test procedure.

4. The arrangement as set forth in claim 2, wherein the output unit is configured to display the results of the evaluation in a form selected from the group consisting of graphic form and a synoptic visual display with a two-dimensional or three-dimensional image of the joint region obtained by an imaging test procedure.

5. The arrangement as set forth in claim 1, wherein first and second adjustable clamping devices are provided as adapters to fix the first and second bone-fixed multipoint transducers in position on the joint and on the extremity, respectively.

6. The arrangement as set forth claim 1, further comprising:
a third bone-fixed multipoint transducer operative to attach in a manner selected from the group consisting of substantially rigid attachment and substantially rigid attachment by means of an adjustable sleeve, to a second extremity which originates from a second hip or shoulder joint that is not undergoing surgery, and
wherein the evaluation unit is configured to determine geometric parameters of the second hip or shoulder joint as a reference for the geometric parameters of the first joint.

7. The arrangement as set forth in claim 1:
wherein the mobile multipoint transducer is configured to provide external sensing of bony references on the second extremity originating from the joint being replaced and from the second hip or shoulder joint; and
wherein the evaluation unit is configured to evaluate the measurement point coordinates of those bony references in order to determine a geometric parameter.

8. The arrangement as set forth in claim 7, wherein the geometric parameter is a length of the extremity.

9. The arrangement as set forth in claim 1, further comprising a second mobile multipoint transducer in the form of a movable sensor to sense bony references on the second extremity originating from the joint being replaced and from the second hip or shoulder joint; and wherein the evaluation unit is configured to evaluate the measurement point coordinates of those bony references in order to determine a length of the extremity.

10. The arrangement as set forth in claim 1, further comprising:
an instrument selected from the group consisting of a resectioning instrument, a milling tool, and a rasp can be rigidly connected to said mobile multipoint transducer or to a further multipoint transducer to form a geometrically calibrated, navigable tool/transducer unit, so that from the transducer signals of that unit there can be determined position coordinates of a part selected from the group consisting of an operational part of the said resectioning instrument, a milling head and a rasp part, and therefrom, position coordinates of a resection zone produced with the resectioning instrument, and
an input interface configured to enter instrument parameters of the resectioning instrument.

11. The arrangement as set forth in claim 1, further comprising:
a navigable setting instrument operative to be rigidly connected to the mobile multipoint transducer or a further multipoint transducer to form a geometrically calibrated tool/transducer unit, so that the transducer signals of that unit can be used to determine position coordinates of an operational part of the setting instrument, and
an input interface configured for entering tool parameters of the setting tool.

12. The arrangement as set forth in claim 1, further comprising:
a probe for probing the medullary canal of the extremity originating from the joint, said probe being configured to be rigidly connected to the mobile multipoint transducer or to a further multipoint transducer to form a geometrically calibrated, navigable probe/transducer unit, so that the transducer signals of that unit can be used to determine the position of the medullary canal axis, and
an input interface configured for entering probe parameters.

13. The arrangement set forth in claim 12, wherein the probe is a medullary canal awl probe.

14. A method of ascertaining function-determining geometric parameters of a joint selected from the group consisting of a joint of a vertebrate, a hip joint of a human being, and a shoulder joint of a human being, in preparation for the installation of an implant selected from the group consisting of a joint replacement implant, a hip socket, a shoulder socket and an associated stem implant, said method comprising:
in a first sequence of measurement steps, bringing a mobile multipoint transducer configured for manual sensing of bony references in the joint region into a first plurality of sensing positions and determining in each sensing position the coordinates of the bony reference in question;
in a second sequence of measurement steps, bringing an extremity ending in the joint, to which extremity a second multipoint transducer is rigidly attached, into a second plurality of rotated positions and subjecting the sets of measurement point coordinates recorded in the rotated positions of the extremity to evaluation in order to determine the coordinates of the center of rotation by carrying out a procedure selected from the group consisting of an iterative procedure, and an iterative adjustment calculation in accordance with the least squares method, and using the coordinates of the bony references and of the center of rotation, determining the geometric parameters in accordance with a predetermined process sequence.

15. The method as set forth in claim 14, wherein the first sequence of measurement steps takes the form of sensing iliac crest references in the vicinity of a hip joint, and coordinates of a further bone-fixed multipoint transducer, namely a multipoint transducer fixed to the iliac crest, are evaluated.

16. The method as set forth in claim 14, wherein the first sequence of measurement steps is carried out with a medullary canal probe inserted into the medullary canal of a femur, the medullary canal wall providing bony references.

17. The method as set forth in claim 14, wherein the predetermined process sequence is in the form of a menu guidance system, including an input step for keyboard entry or voice entry of defined real or virtual points of the joint region and/or position reference vectors between such points within the joint region or from those points to joint-function-relevant points on the extremity outside the joint region or for the data transfer of corresponding data of a three-dimensional image from an evaluation program of an imaging test, and a step of displaying a plan result is included.
